(19) United States
(12) Patent Application Publication
(10) Pub. No.: US 2023/0132840 A1
(43) Pub. Date: May 4, 2023

(54) APPARTUS AND METHOD FOR A MAGNETIC THERAPEUTIC BRACELET

(71) Applicants: Glenn Akhavein, Bradenton, FL (US); Charles Zablotsky, Ft. Lauderdale, FL (US)

(72) Inventors: Glenn Akhavein, Bradenton, FL (US); Charles Zablotsky, Ft. Lauderdale, FL (US)

(21) Appl. No.: 17/965,479

(22) Filed: Oct. 13, 2022

Related U.S. Application Data
(60) Provisional application No. 63/360,639, filed on Oct. 16, 2021.

(51) Int. Cl. A61N 2/00; A61N 2/06

(52) U.S. Cl. CPC A61N 2/006; A61N 2/06

(57) ABSTRACT

An apparatus and method for . . .

### CROSS REFERENCE TO PRIOR APPLICATION

This patent application claims the benefit of U.S. Provisional Application No. 63/360,639 filed on Oct. 16, 2021. The above provisional application is incorporated by reference herein in its entirety.

### FIELD OF THE INVENTION

The present invention is directed generally to the field of therapeutic magnets, and, more particularly, to a method and apparatus for a magnetic therapeutic bracelet used to work with the body's nervous system.

### BACKGROUND OF THE INVENTION

It is well known that magnets placed on particular parts of the body can reduce pain sensation from that part of the body as well as treating “referred pain” from another part of the body. There are studies showing that Static Magnetic Fields, (SMF), can reduce pain from the part of the body that they are placed on especially when using Concentric Alternating Pole Magnet, (CAPM), assemblies. However, it is not always practical to place CAPM assemblies onto the part of the body that is producing a pain sensation and some pain/discomfort does not have a singular origin.

Two examples of this are Dysmenorrhea and Menopause. Dysmenorrhea can express itself in cramping of the abdomen, strain in the lower back, migraines, etc., as well as combinations of any of those. Menopause can express itself in hot flashes, night sweats, headaches, etc., and it also can be combinations of any of those. Both Dysmenorrhea and Menopause are complicated as they play out differently from women to women as do the potential remedies for relieving the discomfort associated with each.

The present invention has been shown to relieve the discomfort associated with both Dysmenorrhea and Menopause. Studies have been conducted that show that the current invention can relieve the discomfort associated with Dysmenorrhea and Menopause and it is believed that it is the interaction of CAPM magnetic fields and the Median nerve, particularly inside the left wrist. A Dysmenorrhea study done with CAPM assemblies place on the lower abdomen found that almost 75% of the women had a meaningful pain reduction. Current testing is showing a greater therapeutic effect by increasing the CAPM's magnetic field strength, increasing the alternating field gradient, and placing the CAPM assembly on the inside of the wrist just below the base of the palm.

The apparatus and method employed by the present invention to reduce the discomfort associated with Dysmenorrhea and Menopause without the use of pain reducing drugs, hormone therapy, or other invasive treatments are truly new, useful, novel, and non-obvious.

### SUMMARY OF THE INVENTION

Accordingly, the present invention is directed to an apparatus comprised of high power permanent magnetic elements arranged to form a CAPM assembly with penetrating magnetic fields whose close proximity to each other results in high magnetic field gradients.

More specifically, the invention relates to the use of concentric alternating pole magnets to help alleviate the discomfort associated with Dysmenorrhea and Menopause. The discomfort from either Dysmenorrhea or Menopause is very difficult to treat without the use of hormone therapy, prescription medication, or OTC medication and because of this difficulty women suffer. The present invention solves this difficult problem by reducing or eliminating the discomfort that women experience without the use of hormone therapy or medications. The present invention uses a magnet or a set of, 2 or more, concentric magnets. The concentric magnets are arranged with alternating poles and are then placed on the inside of the wrist just below the palm and over the median nerve, much like wearing a watch on the inside of the wrist as opposed to the outside of the wrist.

In one embodiment the magnets are permanently mounted in a housing that provides an attachment location for bands that wrap around the wrist and hold the magnets in place. In another embodiment the magnets are attached directly to the wrist bands.

In further embodiments the magnets are held in a housing that allows for the user to use different bands and/or remove the magnets from the housing and attach the magnets directly to a band.

In an embodiment of the present invention the magnetic fields can be reoriented by the user to alter the North and South magnetic fields penetrating the body.

In an embodiment the CAPM magnetic fields are fixed so that the user cannot change the North and South orientation.

In an embodiment the penetrating depth of the magnetic fields can be altered by the user.

In an embodiment of the present invention the CAPM assembly can be held against the user's body with or without the use of an additional housing.

In an embodiment of the present invention the user can change the apparatus used to hold the CAPM magnetic assembly against the body. The CAPM assembly could be replaced by a Liner Alternating Pole Magnet, (LAPM), assembly.

The present invention, including its features and advantages, will become more apparent from the following detailed description with reference to the accompanying drawings.

### BRIEF DESCRIPTION OF DRAWINGS

FIG. 1 is an overview of a Magnetic Therapeutic Bracelet (MTB) with a sliding cover, in accordance with various embodiments of the present invention.

FIG. 2 is an exploded view of an MTB, in accordance with various embodiments of the present invention.

FIG. 3 is a view from the bottom of an MTB, in accordance with various embodiments of the present invention.

FIG. 4A is an exemplary illustration of a view of an MTB with the cover removed and a two-part band in place, in accordance with various embodiments of the present invention.

FIG. 4B is a view of an MTB with the cover removed and a one-part band in place, in accordance with various embodiments of the present invention.

FIG. 4C is a view of a close up of a retainer plate, in accordance with various embodiments of the present invention.

FIG. 5A is a view of a view of a case from the top, in accordance with various embodiments of the present invention.

FIG. 5B is a view of a case from the bottom, in accordance with various embodiments of the present invention.

FIG. 6A is a view of a cover from the top, in accordance with various embodiments of the present invention.

FIG. 6B is a view of a cover from the bottom, in accordance with various embodiments of the present invention.

FIG. 7A is a view of a band prior to dropping it into the case, in accordance with various embodiments of the present invention.

FIG. 7B is a view of a band after it has been dropped into the case, in accordance with various embodiments of the present invention.

FIG. 7C is a view of a band after it has been pulled outward locking it into the case, in accordance with various embodiments of the present invention.

FIG. 8A is a cross section of an MTB with the cover slid partially off, in accordance with various embodiments of the present invention.

FIG. 8B is a detailed view of a locking tab with the cover slid partially off, in accordance with various embodiments of the present invention.

FIG. 9A is a top view of a Magnetic Therapeutic Bracelet, MTB, with a rotating cover, in accordance with various embodiments of the present invention.

FIG. 9B is a bottom view of a Magnetic Therapeutic Bracelet, MTB, with a rotating cover, in accordance with various embodiments of the present invention.

FIG. 10A is a view of a case from the top, in accordance with various embodiments of the present invention.

FIG. 10B is a view of a case from the bottom, in accordance with various embodiments of the present invention.

FIG. 11A is a view of the top of a rotating cover, in accordance with various embodiments of the present invention.

FIG. 11B is a view of the side of a rotating cover, in accordance with various embodiments of the present invention.

FIG. 11C is a view of the bottom of a rotating cover, in accordance with various embodiments of the present invention.

FIG. 11D is a detailed view of a locking tab, chamfer, and indent on a rotating cover, in accordance with various embodiments of the present invention.

FIG. 12A is a view of a rotating cover prior to dropping it onto a case, in accordance with various embodiments of the present invention.

FIG. 12B is a view of a rotating cover after it has been placed onto a case and prior to locking it into place, in accordance with various embodiments of the present invention.

FIG. 12C is a view of a rotating case after it has been locked onto a case, in accordance with various embodiments of the present invention.

FIG. 12D is a view of the rotating case after it have been locked into a case with the top of the cover removed so the interaction between cover and case locking components can be seen, in accordance with various embodiments of the present invention.

FIG. 13A a top view of a Magnetic Therapeutic Bracelet, MTB, with a case that allows the bands to be slid sideways into slots, in accordance with various embodiments of the present invention.

FIG. 13B is an exploded view of a Magnetic Therapeutic Bracelet, MTB, with a case that allows the bands to be slid sideways into slots, in accordance with various embodiments of the present invention.

FIG. 14A is a view of an MTB that does not have a housing for a CAPM assembly, in accordance with various embodiments of the present invention.

FIG. 14B is an exploded view of an MTB that does not have a housing for a CAPM and shows how Velcro or a magnetic backing plate could be used to fix a CAPM to a band, in accordance with various embodiments of the present invention.

FIG. 15A is a view of a CAPM assembly with its corresponding cross section below it showing the magnetic flux lines at the cross-sectional plane, in accordance with various embodiments of the present invention.

FIG. 15B is a view of a LAPM assembly with its corresponding cross section below it showing the magnetic flux lines at the cross-sectional plane, in accordance with various embodiments of the present invention.

FIG. 16A is a top view of a MTB with a case that allows the bands to be attached by using spring pins, in accordance with various embodiments of the present invention.

FIG. 16B is an exploded view of a MTB with a case that allows the bands to be attached using spring pins, in accordance with various embodiments of the present invention.

LIST OF REFERENCE NUMERALS IN DRAWINGS

| | |
|---|---|
| 1. | Magnetic Therapeutic Bracelet (MTB) |
| 2. | CAPM housing |
| 3. | Center magnet |
| 4. | Middle magnet |
| 5. | Outer magnet |
| 6. | Case |
| 7. | Cover |
| 8. | Retaining plate |
| 9. | Band 1 |
| 10. | Band 2 |
| 11. | CAPM assembly |
| 12. | CAPM assembly pocket |
| 13. | Band assembly |
| 14. | Band pin |
| 15. | Band holes |
| 16. | Case bottom |
| 17. | Band lug |
| 18. | One-piece band |
| 19. | Band Velcro |
| 20. | Band anchor |
| 21. | Case top |
| 22. | Case band plane |
| 23. | Case opening |
| 24. | Retaining plate notch |
| 25. | Band lug stop |
| 26. | CAPM assembly mount |
| 27. | Cover slot |
| 28. | Cover exterior portion |
| 29. | Cover interior portion |
| 30. | Cover bottom surface |
| 31. | Cover rail |
| 32. | Cover locking tab |
| 33. | Lock direction indicator |
| 34. | Cover push bumps |
| 35. | Case exterior portion |
| 36. | Case interior portion |
| 37. | Retainer plate tab |
| 38. | Retaining plate lifter |
| 39. | Band lug lock |
| 40. | Locking tab chamfer |
| 41. | Cover rail stop |
| 42. | Band lug retainer |
| 43. | Cover locking tab |
| 44. | Cover locking tab indent |
| 45. | Cover locking tab chamfer |
| 46. | Case locking groove |
| 47. | Cover locking tab stop |
| 48. | One-piece band anchor |
| 49. | Cover locking tab detent |
| 50. | CAPM assembly backer plate |
| 51. | One-piece band |
| 52. | Slide in band slot |
| 53. | No housing embodiment |
| 54. | LAPM assembly |
| 55. | Band Spring Pins |
| 56. | Band Spring Pin Holes |

### DETAILED DESCRIPTION

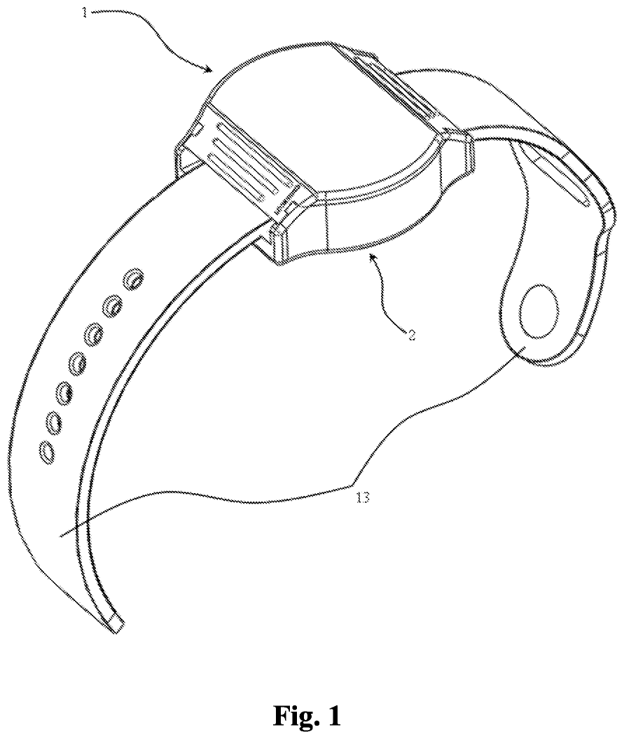

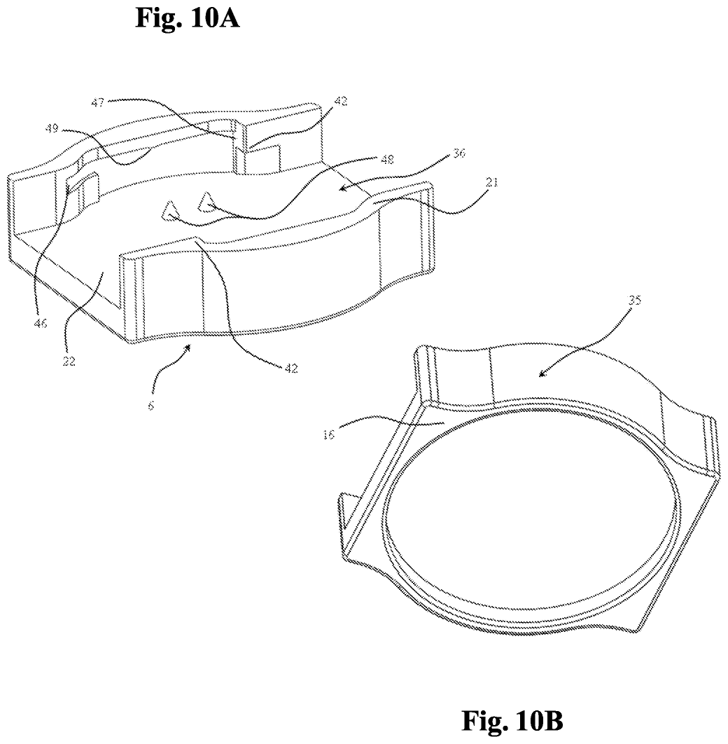

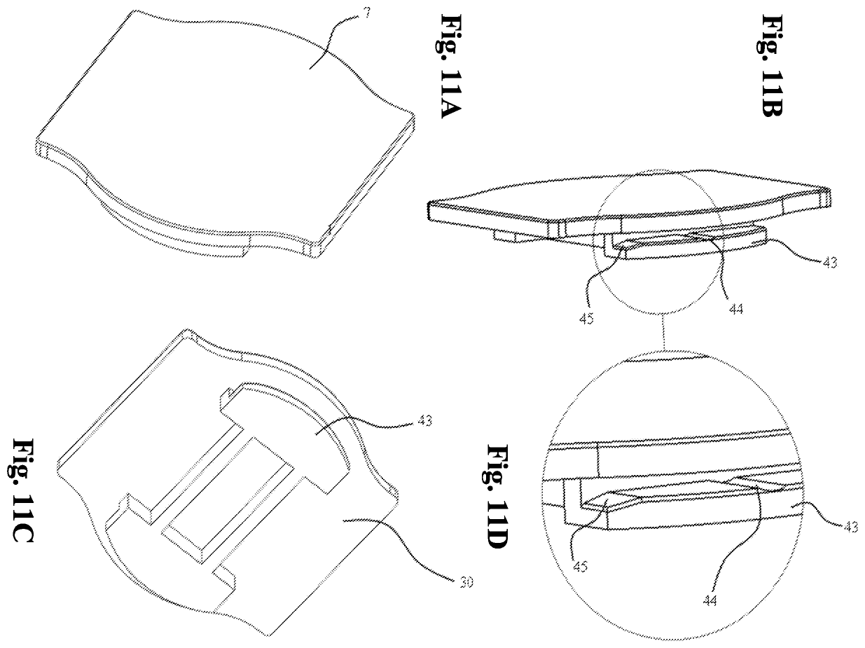

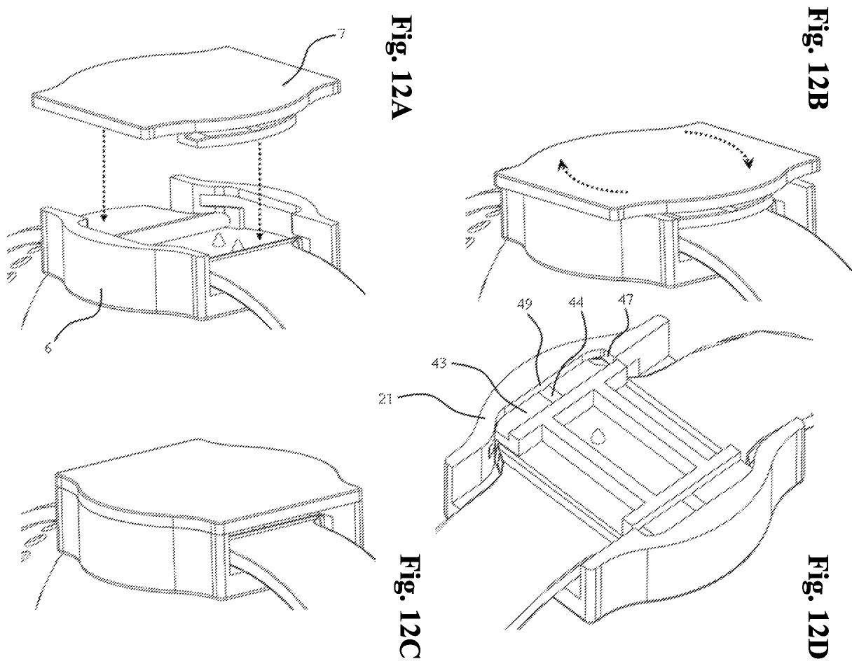

FIGS. 1 through 16 illustrate a method and apparatus for a magnetic therapeutic bracelet used to work with the body's nervous system wherein high-power permanent magnetic elements are arranged to form a CAPM assembly with penetrating magnetic fields whose close proximity to each other results in high magnetic field gradients.

It is expected that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope. A description of embodiments of the present invention will now be given with reference to the Figures.

Referring now to FIG. 1, in which a a preferred embodiment of Magnetic Therapeutic Bracelet (MTB), 1 is made up of two primary components; CAPM housing 2 and wrist bands 13.

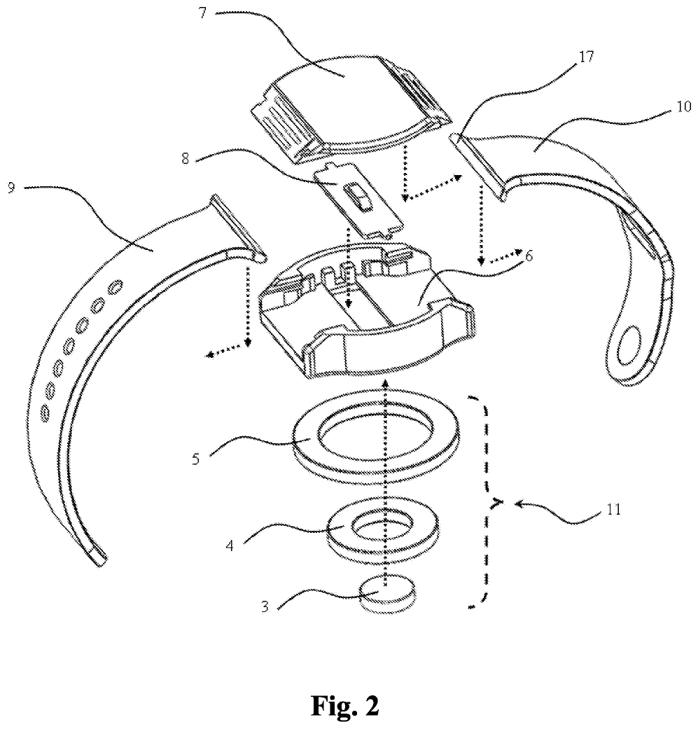

FIG. 2 illustrates how CAPM housing 2 holds CAPM assembly 11, which is made up of three alternating pole concentric magnets; center magnet 3, middle magnet 4, and outer magnet 5. By virtue of the fact that center magnet 3, middle magnet 4, and outer magnet 5 have alternating poles, they will lock themselves into a concentric arrangement and will then act as a single component, CAPM assembly 11, which due to the high magnetic strength of the individual magnets that make up CAPM assembly and their close proximity to one another, become very difficult to separate.

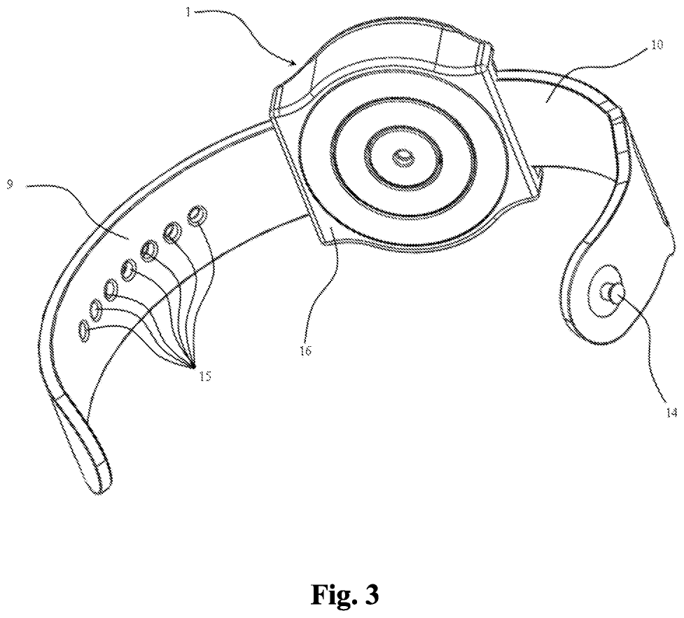

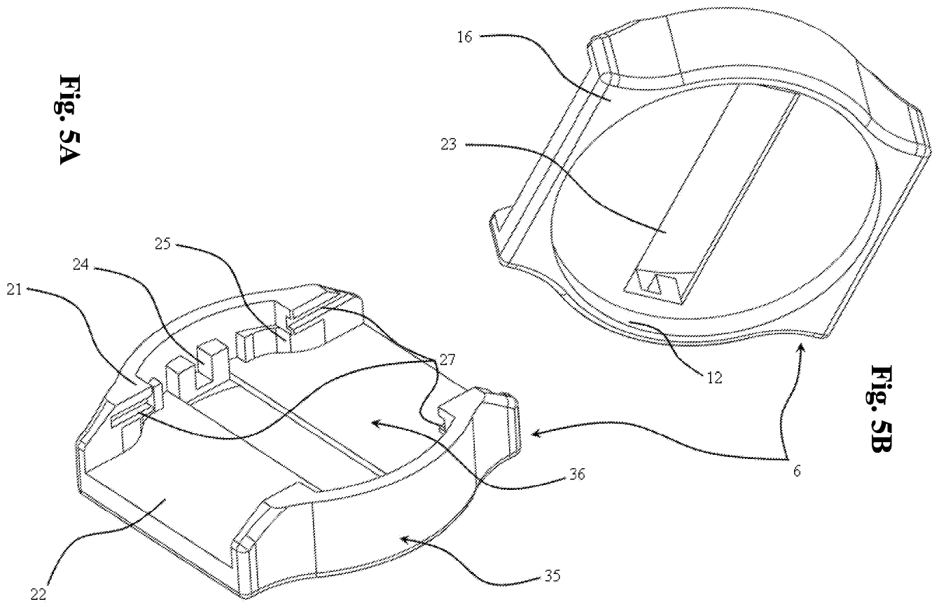

It can be seen in FIG. 3 and FIG. 5B how CAPM assembly 11 fits into CAPM assembly pocket 12 which is in the bottom of case 6. Once CAPM assembly 11 is placed into CAPM assembly pocket 12 retaining plate 8 is inserted into case 6 from the top and the magnetic attraction between the retaining plate 8 and CAPM assembly 11 holds CAPM assembly 11 in case 6. An alternate assembly method would be to not insert CAPM assembly 11 all at once, but instead, insert outer magnet 5, then retaining plate 8, then middle magnet 4, and finally center magnet 3. Variations of this assembly would be considered obvious to one skilled in the art.

Case 6 is usually held against the user's wrist by either a one or two-part band similar to how a watch is worn on the inside of the wrist instead of the outside. The band has a portion that is fixed to case 6 and uses a fixture method, similar to existing watch bands, and the free ends attach to each other using such things as a pin and holes, a buckle and holes, a clasp, Velcro, or by the use of magnets in the band. These and other methods are clearly known to a person skilled in the art.

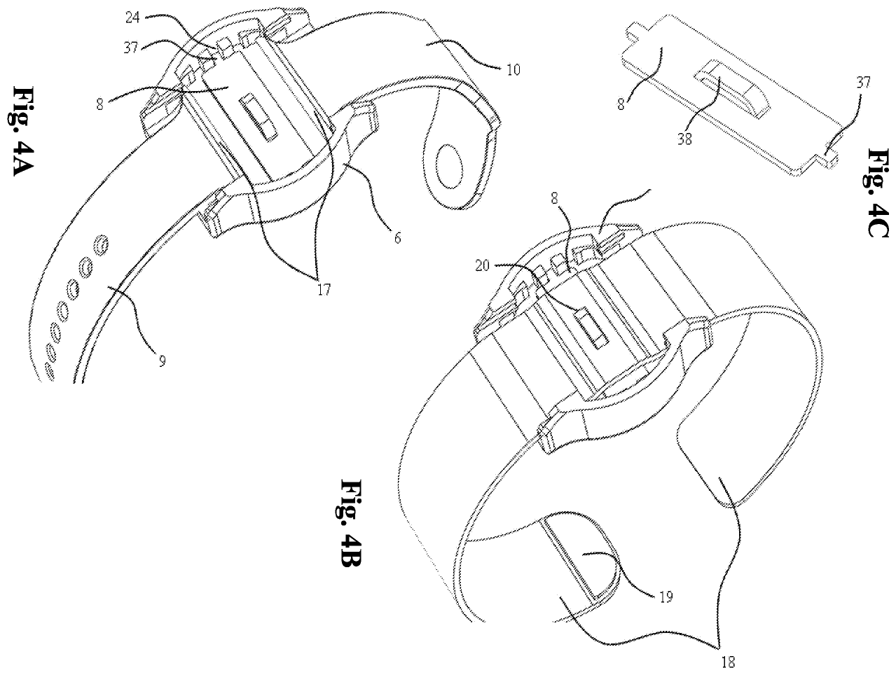

In FIG. 4B, if the band is a single component such as one-piece band 18, the middle portion of it is that is held in case 6, band anchor 20, helps keep one-piece band 18 fixed to case 6. In this embodiment the band anchor 20 is an opening in one-piece band 18 that fits over retaining plate lifter 38 as illustrated in FIG. 4C. The ends of one-piece band 18 are then held together with a fixture method such as band Velcro 19. If the band is not a single component such as band assembly 13, then it is usually made up of two components, band 9 and band 10, which employ both an apparatus and method to attach their free ends to each other, such as the fixture methods mentioned above, and the fixed ends to case 6. In one embodiment, band 10 and band 9 employ attachment apparatus band pin 14 which mates up to band holes 15 as illustrated in FIG. 3. FIG. 4A also illustrates how Band 9 and band 10 each have band lug 17 on the end that allows for them to be fixed to case 6.

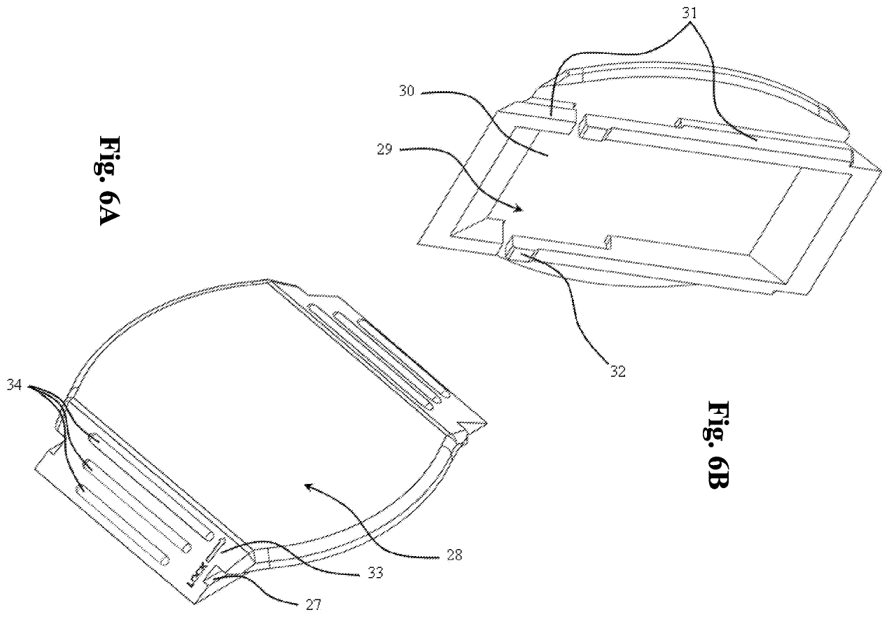

CAPM housing 2 is made up of case 6 and cover 7. Cover 7 has an exterior portion 28 with multiple features and an interior portion 29 with multiple features as illustrated in FIGS. 6A and 6B. Case 6 has a case top 21 and a case bottom 16. Case 6 has and exterior portion 35 and interior portion 36 as illustrated in FIG. 5A and FIG. 5B. Below case top 21 on interior portion 36 of case 6 is case band plane 22. This is where one-piece band 18, or if using band assembly 13, band lugs 17 come into contact with case 6. There is a CAPM assembly pocket 12 on the bottom of case 6 that is recessed from case bottom 16 which is capable of encompassing CAPM assembly 11. CAPM assembly pocket 12 is sized so that the magnets in CAPM assembly 11 are always even or protrude slightly beyond case bottom 16 so that when placed on the wrist the magnets in CAPM assembly 11 are ensured of touching the user's wrist. It is obvious that CAPM assembly pocket 12 could be designed so as to allow CAPM assembly 11 to sit recessed into CAPM assembly pocket 12, which would keep CAPM assembly 11 from contacting the user's wrist; however, testing has shown that to be an inferior design.

When CAPM assembly 11 is placed into CAPM assembly pocket 12, retaining plate 8 is put into case 6 from the top with retaining plate tab 37 fitting into retaining plate notch 24 as illustrated in FIG. 4A. Retaining plate 8 is lowered until the bottom of retaining plate 8 comes into contact with case band plane 22. At this point the magnetic attraction between CAPM assembly 11 and retaining plate 8 is strong enough to hold both components in place. When the user wants to remove or rearrange CAPM assembly 11 from case 6, the user lifts up on retaining plate 8 via retaining plate lifter 38 at which time CAPM assembly 11 is free to be removed from case 6 by dropping out of CAPM assembly pocket 12 once retaining plate 8 has been removed to a degree where there is no longer a strong enough magnetic attraction between CAPM assembly 11 and retaining plate 8. If CAPM assembly 11 fits tightly into CAPM assembly 12 pocket, then the user may push CAPM assembly 11 out of CAPM assembly pocket 12 from the top via case opening 23 as illustrated in FIG. 5B.

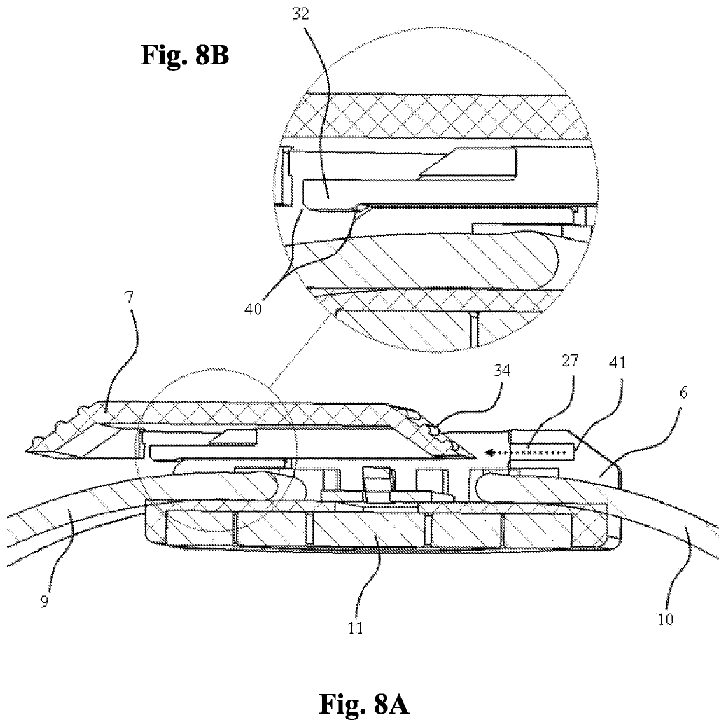

To access retaining plate 8 or to remove either one-piece band 18 or band assembly 13 cover 7 must first be removed. To do this the user pushes laterally on cover push bumps 34 so that cover 7 slides laterally until cover rails 31 have slid past cover slots 27 as illustrated in FIG. 8A and FIG. 8B. Initially cover 7 is held in place on case 6 by locking tabs 32. Locking tabs 32 have locking tab chamfers 40 that engage with mating chamfers on case 6 thus keeping cover 7 in place. When cover 7 is pushed laterally with enough force, locking tab chamfers 40 push against their mating chamfers on case 6 which causes locking tabs 32 to flex upwards into cover slot 27 thus allowing cover 7 to slide laterally. Once cover 7 has cleared case 6 it can be set aside while the user either replaces the bands or removes retaining plate 8 to remove CAPM assembly 11.

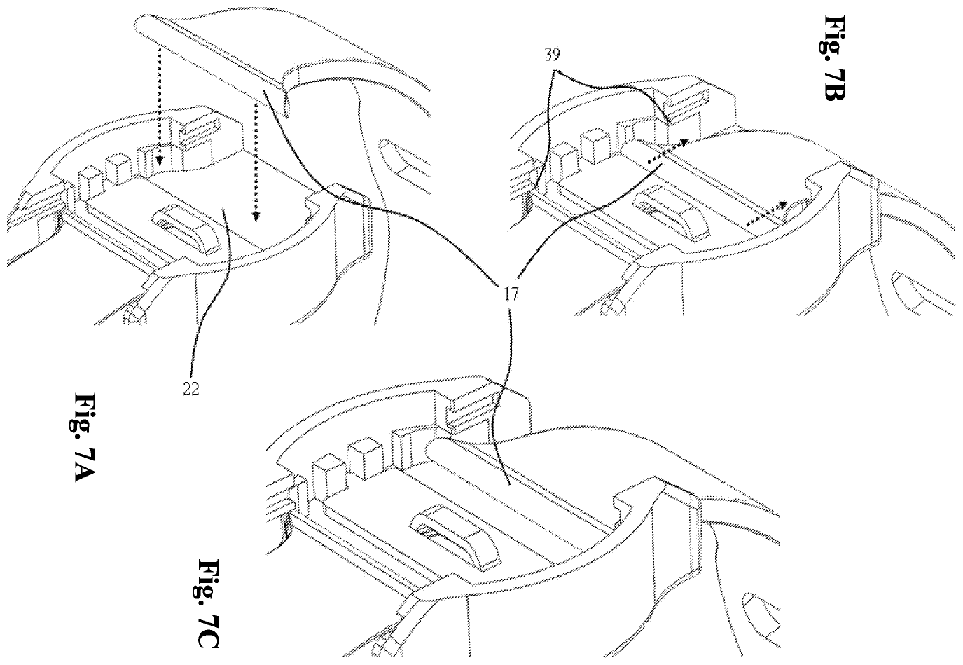

When the user wants to reassemble MTB 1, the user first places CAPM assembly 11 back into case 6 and places retaining plate 8 back into case 6 which keeps them both in place. Then band 9 is dropped into case 6 from the top until band lug 17 comes in contact with case band plane 22 at which time band 9 is slid away from the center of case 6 until band lugs 17 come into contact with band lug stops 25 and are then kept in place by band lug locks 39 as illustrated in FIG. 7A, FIG. 7B, and FIG. 7C. Once band 9 is in place band 10 is installed into case 6 in the same manner. The order of installing band 9 or band 10 is not critical. If a one-piece band is being used, then it is just dropped straight down into case 6 so that band anchor 20 drops over retainer plate 8 as illustrated in FIG. 4A. Regardless of which band style is used, the next step in reassembly is to replace cover 7. This is done by aligning cover rails 31 with cover slots 27 and then pushing laterally against cover push bumps 34 in the direction of the lock direction indicator 33 until cover rail 31 hits cover rail stop 41 which coincides with locking tab chamfers 40 dropping back down against the mating chamfers in case 6.

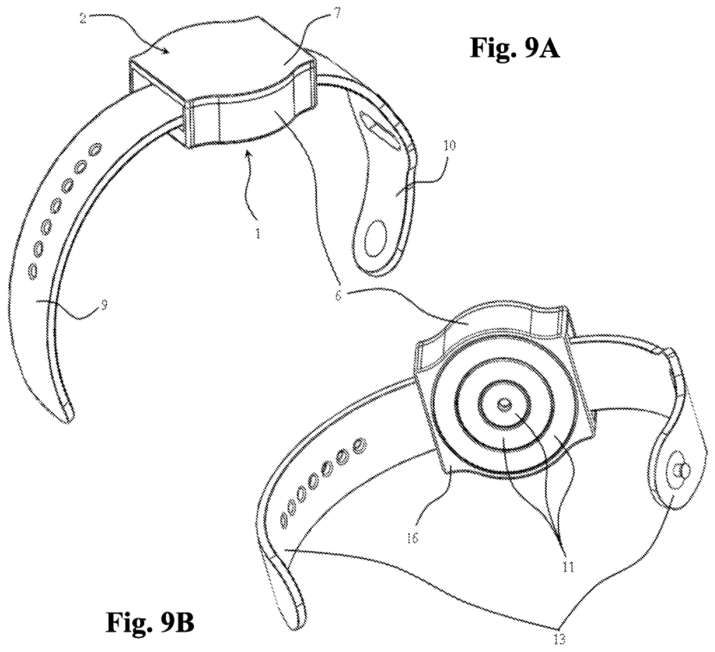

In another embodiment of the present invention, MTB 1 has case 6 which is designed to have cover 7 lock into place with a rotating motion instead of a sliding motion as illustrated in FIG. 9A through FIG. 12D. Additionally, this embodiment is designed without CAPM assembly pocket 12 as case 6 is molded around CAPM assembly 11. In this embodiment CAPM assembly 11 is now permanently part of case 6 and cannot be rearranged by the end user. It is important to note that the sliding cover embodiment and the rotating cover embodiment could be made with an insert molded CAPM assembly 11, a glued-in CAPM assembly 11, or a removable CAPM assembly 11. In either case CAPM assembly 11 should be level with case bottom 16 or slightly protruding from case bottom 16 so that CAPM assembly 11 is always in contact with the user's wrist.

In the rotating cover embodiment cover 7 has locking tabs 43 that are designed to engage with case locking grooves 46. To assemble cover 7 to case 6 cover 7 is rotated approximately 90 degrees from its normal position and then lowered onto case 6 until cover bottom surface 30 comes into contact with case top 21. Then when cover 7 is rotated cover locking chamfers 45 slide into case locking grooves 46 until cover locking tabs 43 come into contact with cover locking tab stops 47. While cover 7 is being rotated into the locked position cover locking tab detent 49 is pushing down on dover locking tab 43 causing it to flex slightly. This happens until cover locking tab 43 comes into contact with cover locking tab stop 47 at which time cover locking tab detent 49 is now lined up with cover locking tab indent 44 which then holds cover 7 in place.

To remove cover 7 from case 6 the user simply rotates cover 7 back approximately 90 degrees until cover locking tabs 43 clear case locking grooves 46 and cover 7 can now be lifted off of case 6. The process for inserting band 9 and band 10 are similar to what has already been discussed except in this embodiment band lugs 17 are held in place by band lug retainer 42. When using one-piece band 18 with this embodiment one-piece band 18 is held in place within case 6 by one-piece band anchors 48.

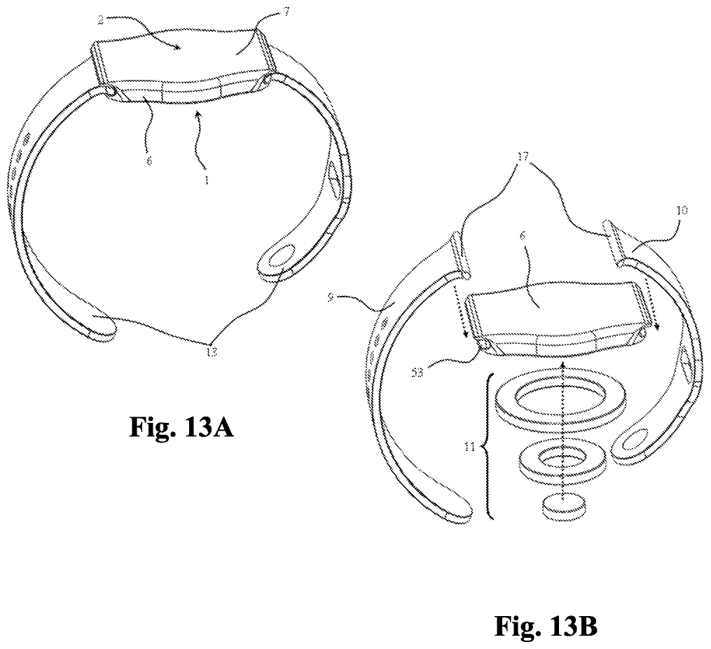

In another embodiment of the present invention, MTB 1 has case 6 but does not have cover 7 as illustrated in FIG. 13A and FIG. 13B. Additionally, this embodiment is designed without CAPM assembly pocket 12 as case 6 is molded around CAPM assembly 11 or CAPM assembly 11 is glued into CAPM assembly pocket 12. In this embodiment CAPM assembly 11 is now permanently part of case 6 and cannot be rearranged by the end user. In this embodiment case 6 has slide in band slots 53 that are designed so that band lugs 17 of band 9 and band 10 slide into slide in band slots 53 and are then held in place. In this embodiment CAPM assembly 11 should be level with case bottom 16 or slightly protruding from case bottom 16 so that CAPM assembly 11 is always in contact with the user's wrist. It is important to note that even though this embodiment is best accomplished with CAPM assembly 11 molded into or glued into case 6, CAPM assembly 11 could be held in place by placing a plate, such as CAPM assembly backer plate 50 on top of case 6 thus holding CAPM assembly 11 in place or molding CAPM assembly backer plate 50 into case 6.

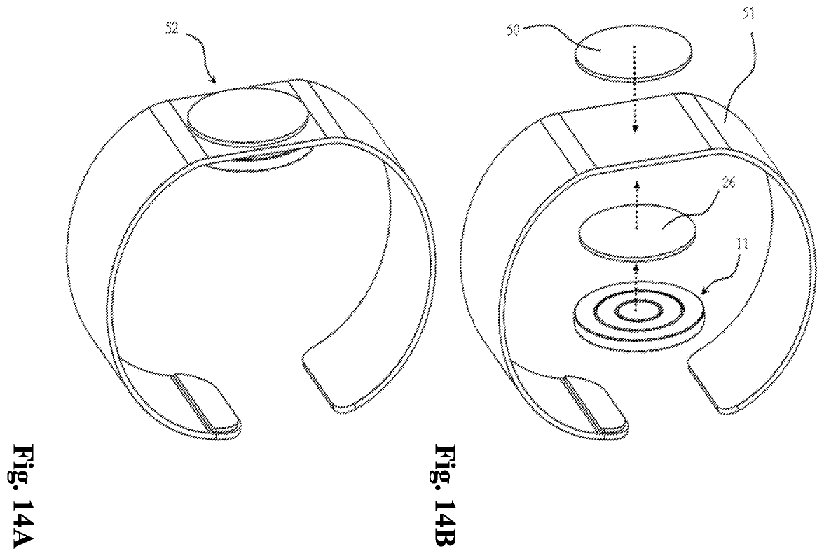

In another embodiment illustrated in FIG. 14A and FIG. 14B, no housing embodiment 52, MTB 1 does not have CAPM housing 2. In this simplified embodiment, CAPM assembly 11 is held onto one-piece band 51 by various means. CAPM assembly 11 can have a mounting apparatus that holds it to one-piece band 51 such as CAPM assembly mount 26. In this embodiment CAPM assembly mount 26 could be a piece of Velcro that has adhesive on one side and then the Velcro is placed on the underside of one-piece band 51 and will cling to one-piece band 51. In this embodiment, CAPM assembly 11 is a fixed assembly that is not rearrangeable by the user.

In another embodiment illustrated in FIG. 14A and FIG. 14B, CAPM assembly 11 is placed on the underside of one-piece band 51 and CAPM assembly backer plate 50 is placed on the top side of one-piece band 51. In this embodiment one-piece band 51 is sandwiched between CAPM assembly 11 and CAPM assembly backer plate 50, and the user does have the ability to remove CAPM assembly 11 and rearrange it as the entire assembly is held together by the magnetic attraction of CAPM assembly 11 and CAPM assembly backer plate 50.

It is important to note that if CAPM assembly 11 is removably housed in an embodiment that employs a CAPM housing 2, CAPM assembly 11 may be removed from CAPM housing 2 and then with the use of backer plate 50 affixed to one-piece band 51. It is also important to note that if CAPM assembly 11 is affixed to one-piece band 51, CAPM assembly 11 may be removed from one-piece band 51 and inserted back into CAPM housing 2.

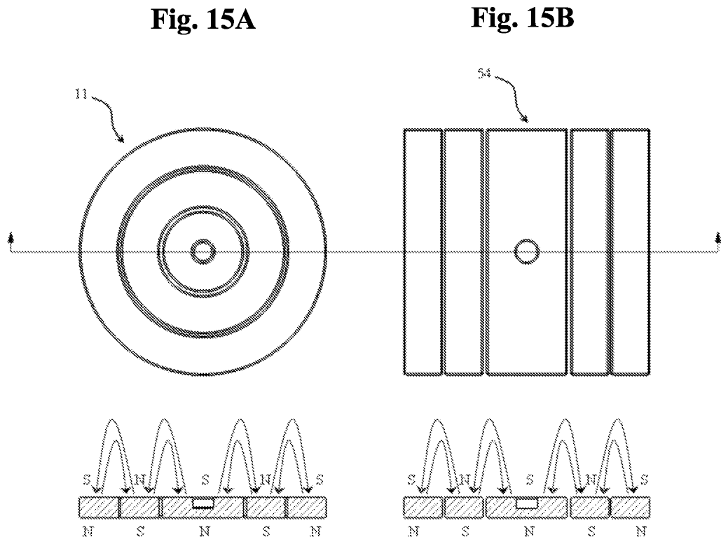

In another embodiment of the present invention illustrated in FIG. 15A, CAPM assembly 11 could be substituted with LAPM assembly 54. FIG. 15A illustrates how the magnetic field taken at the cross-sectional line in FIG. 15A is the same for both CAPM assembly 11 and LAPM assembly 54 as illustrated in FIG. 15B. The equivalent to CAPM assembly 11 or LAPM assembly 54 could be obtained by taking a singular magnet and magnetizing it with a plurality of alternating poles.

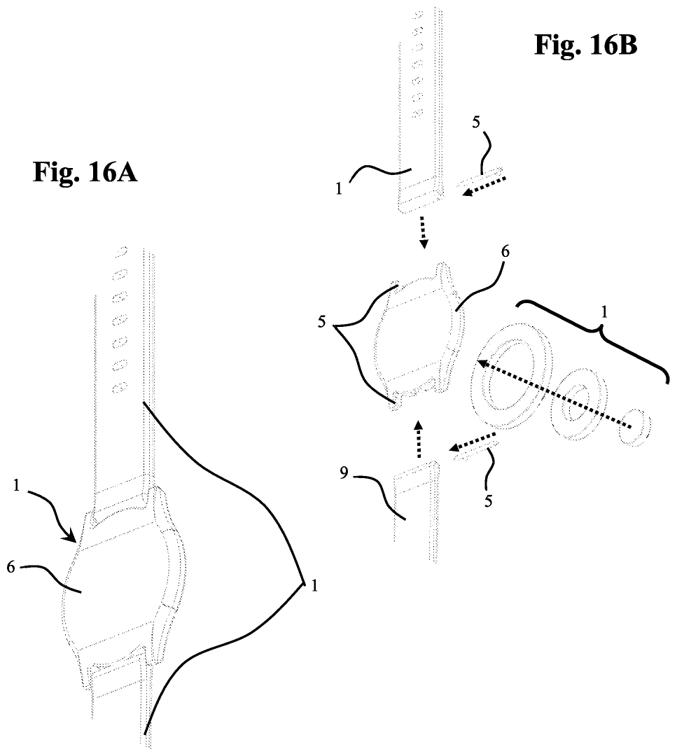

In another embodiment of the present invention, MTB 1 has case 6 but does not have cover 7 as illustrated in FIG. 16A and FIG. 16B. This embodiment is similar to what is shown in FIG. 13A and FIG. 13B except that the band 9 and band 10 are attached to case 6 via spring pins 55 which lock into spring pin holes 56 that are in case 6.

The foregoing description comprises illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method. Many modifications and other embodiments of the invention will come to mind to one skilled in the art to which this invention pertains having the benefit of the teachings presented in the foregoing descriptions. Although specific terms may be employed herein, they are used only in generic and descriptive sense and not for purposes of limitation. Accordingly, the present invention is not limited to the specific embodiments illustrated herein.

What is claimed is:

1. An apparatus for a Magnetic Therapeutic Bracelet (MTB) comprising;
a magnet;
an attaching apparatus capable of attaching to said magnet and to a human's arm;
said magnet being of such strength and design so as to interact with a human's median nerve.

2. The MTB of claim 1, wherein said magnet can be a plurality of magnets arranged with alternating poles forming a magnetic assembly.

3. The MTB of claim 1, wherein said magnet can be a singular magnet with a singular north and south pole or said magnet can be a singular magnet that has been magnetized with a plurality of alternating poles.

4. The MTB of claim 1, wherein said magnet can have its magnetic field altered by the addition of a magnetic field altering apparatus.

5. The MTB of claim 1, wherein said attaching apparatus can be a singular apparatus such as a one-piece elastic band or an apparatus made of a plurality of parts such as a two-piece band.

6. The MTB of claim 1, wherein said attaching apparatus can employ the use of a housing in conjunction with said one-piece or two-piece bands thus forming said attaching apparatus.

7. The attaching apparatus of claim 6, wherein said housing may be designed so said magnet is permanently attached to said housing or said magnet may be removably attached to said housing.

8. The attaching apparatus of claim 6, wherein said housing may be designed so said attaching apparatus is permanently attached to said housing or said attaching apparatus may be removably attached to said housing.

9. The attaching apparatus of claim 6, wherein said magnet may be removed from one said attaching apparatus and attached to a different embodiment of said attaching apparatus.

10. A method of assembling and using a Magnetic Therapeutic Bracelet to interact with a human's median nerve comprising:
affixing a magnet to an attaching apparatus; using said apparatus to affix said magnet to a human's arm so as to interact with said human's median nerve.
